(19) United States
(12) Patent Application Publication
(10) Pub. No.: US 2009/0145452 A1
(43) Pub. Date: Jun. 11, 2009

(54) DEVICES AND METHODS OF USE FOR TREATMENT OF SKIN AND HAIR

(75) Inventors: Daniel Griffith Anderson, Sudbury, MA (US); David Thomas Puerta, Melrose, MA (US); Betty Yu, Cambridge, MA (US); Susan Eilidh Bedford, Carlisle, MA (US)

Correspondence Address: FITZPATRICK CELLA HARPER & SCINTO, 30 ROCKEFELLER PLAZA, NEW YORK, NY, 10112, US

(73) Assignee: Living Proof, Inc., Cambridge, MA (US)

(21) Appl. No.: 12/300,456

(22) PCT Filed: May 11, 2007
(86) PCT No.: PCT/US07/68758

§ 371 Date: Jan. 26, 2009

Related U.S. Application Data
(60) Provisional application No. 60/799,825, filed on May 11, 2006.

(51) Int. Cl. A61K 8/72

(52) U.S. Cl. 132/202

(57) ABSTRACT

Devices and methods of use are disclosed for treating hair and/or skin. The devices include light sources or heat sources useful in activating polymerization initiators thereby polymerizing monomers in situ on skin or hair. Exemplary devices include hair brushes, eyelash brushes, curling irons, curlers, and flat irons.

### RELATED APPLICATIONS

The present application claims priority under 35 U.S.C. § 119(e) to U.S. provisional patent application, U.S. Ser. No. 60/799,825, filed May 11, 2006; which is incorporated herein by reference.

### BACKGROUND OF THE INVENTION

Hair treatments, cosmetics, and related accessories are widely used by men and women worldwide. Hair treatments, which include but are not limited to, hairsprays, mousses, or gels, hold hair in a particular configuration, color hair, add vitamins or other nutrients to hair, and/or otherwise alter the look or feel of hair according to the choices of the stylist. Cosmetics, which include creams, lotions, or powders, hide imperfections or blemishes on skin, color skin, alter the tone of skin, or otherwise alter the look or feel of skin according to the choices of the stylist. More recently, certain cosmetic products also remove or reduce wrinkles on skin, and/or slow, stop, or reverse the wrinkling process that occurs as a result of, for example, aging or sun exposure.

However, existing hair treatments and cosmetics suffer from numerous limitations. By way of example, existing hair treatments are not robust and can lose efficacy during the course of a user's daily routine. For instance, existing hair treatments can lose their efficacy when exposed to water through, for example, washing or ambient humidity. Many existing hair treatments also weigh down hair, flake, leave unsightly residues, do not dry quickly, do not set quickly, do not provide adequate hold, and are often not effective for hard-to-treat hair, such as naturally curly hair. Similarly, cosmetics lose effect and, in some cases, smear when exposed to water through, for example, washing, humidity, tears, sweat, or saliva (i.e., in the case of lipstick). Additionally, many cosmetics which purport to treat wrinkles are not effective for that purpose.

Thus, there exists a need for hair treatments and cosmetics which withstand the rigors of a typical user's daily routine, and in particular can maintain efficacy in a variety of environments, e.g., a variety of climates with increased humidity and temperature levels. It is preferable that the hair treatments do not excessively weigh down hair, flake, or leave undesirable residues. It is desirable that the hair treatments dry and set relatively quickly, provide adequate hold, and treat even hard-to-treat hair without undue damage or toxicity. It is desirable that the cosmetics also maintain efficacy in a variety of environments and when exposed to a variety of conditions, again without causing undue damage or toxicity. For example, preferably, the cosmetics can hide, lessen, or remove wrinkles, and/or slow, stop, or reverse the wrinkling process.

### SUMMARY OF THE INVENTION

This invention, in various embodiments, addresses the above-mentioned deficiencies, and others, in the prior art by providing systems, methods, devices, kits, and treatments for hair treatment and cosmetics. The methods generally include at least two phases: in a first phase, a stylist applies the hair treatment to hair (or the cosmetic to skin as the case may be); and in a second phase, the stylist activates the hair treatment (or cosmetic) by, for example, applying light or heat to the hair treatment (or cosmetic). Activation typically causes the polymerization or cross-linking of the applied treatment on the subject's hair or skin via a free radical chemical reaction. In the case of hair treatment, the methods may include an additional phase that typically, though not exclusively, takes place before or after application of the hair treatment, wherein the stylist configures the hair according to a desired hair style.

In various embodiments, the hair treatment or cosmetic is activated in situ (i.e., while the hair treatment is on or in the hair, or while the cosmetic is on the skin). The hair treatment (or cosmetic) is formulated such that when activated, it produces or enhances the desirable properties described above. Thus, aspects of the invention include hair treatment formulations and cosmetic formulations that produce desirable properties when activated, activators that activate the hair treatments and/or the cosmetics, as well as devices for activating the hair treatment or cosmetic, and kits that combine the formulations, activators and/or devices and methods for use thereof.

For example, the hair treatments or cosmetics, in various embodiments, initially (i.e., before being applied to hair or skin) include monomers, oligomers, or polymers (e.g., low molecular weight, or uncross-linked polymers) in a solution, suspension, or gel. The solution may also include an initiator, such as a polymerization initiator, that, upon exposure to an external activator such as light or heat, initiates the activation of the monomers; said initiator can also be separately contained and applied to the hair or skin. After a stylist applies the hair treatments or cosmetics, the stylist uses an external activator, such as a light source or a heat source, to activate the initiator. This activation process can include, for example, polymerizing and/or cross-linking the monomers to generate a polymer in situ on the hair or skin. The hair treatments and cosmetics are formulated such that formed polymers impart desirable properties to hair or skin. This process is particularly effective because in situ activation of the hair treatments or cosmetics further enhances these desirable properties. Particular compositions and processes for use include, but are not limited to, the processes and compositions disclosed in U.S. provisional applications, U.S. Ser. No. 60/793,821, filed Apr. 21, 2006; U.S. Ser. No. 60/798,572, filed May 8, 2006; U.S. Ser. No. 60/800,142, filed May 11, 2006; U.S. Ser. No. 60/800,143, filed May 11, 2006; U.S. Ser. No. 60/800,146, filed May 11, 2006; and U.S. Ser. No. 60/853,612, filed Oct. 23, 2006; and U.S. non-provisional application, U.S. Ser. No. 11/734,425, filed Apr. 12, 2007; and international PCT applications, PCT/US07/09083, filed Apr. 12, 2007; and PCT/US07/(Attorney Docket Number 2007355-0015, filed May 11, 2007; each of which is incorporated herein by reference. The devices disclosed herein are also suitable for use in conjunction with other methods in the art. For example, the devices may also be useful in the treatment of skin diseases such as psoriasis or skin cancer. The light or heat for activation of the hair treatment can be provided by traditional hair care accessories, traditional hair care accessories supplemented with a light source or heat source, or other devices suitable for emitting radiation (e.g., providing light or heat) as described herein. The external activators can emit light and/or heat simultaneously with the application of the hair treatment or cosmetic by the stylist (including self-styling).

Additionally or alternatively, the external activators can emit light and/or heat after the stylist or user applies the hair treatment or cosmetic. In certain embodiments, a stylist or user can use a single device to both apply the hair treatment or cosmetic and to emit the light and/or heat. In some embodiments, the light and heat can be emitted simultaneously or sequentially, and the device may emit heat without light or light without heat.

In one aspect, the invention includes a method for treating hair or skin comprising applying a hair treatment or cosmetic to the hair or skin, respectively, and activating said hair treatment or cosmetic with an external activator such as light and/or heat comprising one or more activating devices as described herein

In another aspect, the invention relates to the activating devices as described herein. In one embodiment the invention includes a system for activating a hair treatment applied to hair, comprising a plurality of elongate cylindrical curlers for wrapping respective tresses of hair thereabout, wherein the elongate cylindrical curlers include respective activators for activating the hair treatment. In certain embodiments, the curlers heat the treated hair thereby activating the applied polymerization initiators and causing the polymerization of the applied monomers. In certain embodiments, the curlers are used to apply light to the treated hair thereby activating the applied polymerization initiators and causing the polymerization of the applied monomers. In certain embodiments, the curlers apply both light and heat to the treated hair.

In another aspect, the invention includes a device for activating a hair treatment applied to hair, comprising an elongate body, an elongate handle at an angle with respect to the elongate body, and a light source for emitting light to activate the hair treatment. The light source may be an IR, visible, or UV light source. The wavelength(s) of light generated by the light source should typically correspond with the wavelength of light for activating the polymerization initiator used in the hair treatment. The light source may allow for generation of light of varying wavelengths and intensity. Varying the output of light allows for greater control of the polymerization process. In certain embodiments, the light source is an IR light source. In other embodiments, the light source is a visible light source. In still other embodiments, the light source is a UV light source. In certain embodiments, the light source emits light with a wavelength of about 200 nm to about 600 nm and an intensity of about 500 μW/cm² to about 10,000 μW/cm². In certain particular embodiments, the light source emits light at a wavelength of 365 nm and at an intensity of about 7,000 μW/cm². In certain embodiments, the light source emits light at an intensity of about 4000, 5000, 6000, 7000, 8000, or 9000 μW/cm². In certain embodiments, the light source emits light at a wavelength of about 200 to about 400 nm.

In another aspect, the invention includes a device for activating a hair treatment applied to hair, comprising a handle, a body affixed to the handle, and a plurality of bristles emanating from the planar surface for brushing the hair and for emitting light and/or heat to activate the hair treatment.

In another aspect, the invention includes a device for activating a hair treatment applied to hair, comprising a comb with a plurality of teeth for combing the hair and for emitting light and/or heat to activate the hair treatment.

In another aspect, the invention includes a device for activating a hair treatment or cosmetic applied to hair or skin, respectively, comprising a finger cover for covering at least a portion of a stylist's or user's finger(s), and an activator for activating the hair treatment or cosmetic. In certain embodiments, the device is a glove for activating the hair or cosmetic treatment.

In another aspect, the invention includes a device for activating a hair treatment or cosmetic applied to hair or skin, respectively, comprising a container for storing and/or dispensing the hair or cosmetic treatment, and an activator integral to the container for activating the hair or cosmetic treatment.

In another aspect, the invention includes a cosmetics applicator, comprising an applicator for applying the cosmetics to a target region of skin or hair, and an activator for activating the cosmetics.

In another aspect, the invention includes a system for activating a hair treatment or cosmetic applied to the hair or skin, respectively, comprising a housing for encapsulating at least a portion of the body, and one or more activators coupled to the housing for activating the hair or cosmetic treatment.

### DEFINITIONS

As used herein, the singular forms “a”, “an”, and “the” include the plural reference unless the context clearly indicates otherwise. Thus, for example, a reference to “a monomer” includes a plurality of such monomers.

“Animal”: The term animal, as used herein, refers to humans as well as non-human animals, including, for example, mammals, birds, reptiles, amphibians, and fish. Preferably, the non-human animal is a mammal (e.g. a rodent, a mouse, a rat, a rabbit, a monkey, a dog, a cat, a primate, or a pig). An animal may be a domesticated animal. In certain embodiments, the animal is human. An animal may be a transgenic animal.

“Biocompatible”: The term “biocompatible”, as used herein is intended to describe compounds that are not toxic to cells. Compounds are “biocompatible” if their addition to cells in vitro results in less than or equal to 20% cell death. The administration in vivo does not cause cancer, birth defects, neurotoxicity, or other such adverse side effects.

“Biodegradable”: As used herein, “biodegradable” compounds are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). The components preferably does not cause inflammation, cancer, birth defects, neurotoxicity, or other such adverse side effects in vivo. In certain preferred embodiments, the chemical reactions relied upon to break down the biodegradable compounds are uncatalyzed. For example, the inventive materials may be broken down in part by the hydrolysis of the ester bonds found in cross-linked material.

“Keratin”: The term “keratin” as used herein refers any one of a class of fibrous structural proteins found in skin. Keratin proteins contains a large quantity of cysteine residues. The helical keratin molecules twist around each other to form elongated strands called intermediate filaments.

“Monomer”: As used herein, a “monomer” is a chemical compound that is linked to other monomers covalently to form a polymer. Examples of monomers include acrylates, methacrylates, epoxide containing compounds, styrenes, and vinyl alcohol. In certain embodiments, the monomers useful in accordance with the present invention are susceptible to free radical polymerization.

“Oligomer”: The term “oligomer,” as used herein, refers to a chemical compound with a finite number of structural units connected by covalent bonds. An oligomer has less monomeric units than the corresponding polymer. An oligomer typically has between 3 to 100 monomeric units making up its structure. In certain embodiments, less than 10 monomeric units are found in the oligomer. In certain embodiments, less than 20 monomeric units are found in the oligomer. In certain embodiments, less than 50 monomeric units are found in the oligomer. In certain embodiments, less than 100 monomeric units are found in the oligomer.

“Peptide” or “protein”: As used herein, a “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polymer”: The term “polymer,” as used herein, refers to a chemical compound of repeating structural units (monomers) connected by covalent bonds. A polymer is typically of high molecular weight and may comprise 10 s to 100 s to 1000 s or even more monomers. In certain embodiments, the polymer comprises at least 10 monomeric units linked covalently together. In certain embodiments, the polymer may be a co-polymer comprising different types of polymers. The polymer may be cross-linked or uncross-linked. The polymer may be linear or branched. In certain embodiments, the polymer is formed by in situ polymerization on hair.

### BRIEF DESCRIPTION OF THE DRAWING

These and other features and advantages will be more fully understood by the following illustrative description with reference to the appended drawings, in which like elements are labeled with like reference designations, and in which fluidic connections are intended to represent the interrelation of the connections and components but may not depict the actual physical location of the connections and components. The drawings may not be drawn to scale.

FIG. 1 shows a curling iron 100 according to an illustrative embodiment of the invention.

FIG. 2 shows a flat iron 200 according to an illustrative embodiment of the invention.

FIG. 3 shows hair curlers 302a-c according to an illustrative embodiment of the invention.

FIG. 4 illustrates an external activator 400 configured like a blow-dryer, according to an illustrative embodiment of the invention.

FIG. 5 shows a hair brush 500 according to an illustrative embodiment of the invention.

FIGS. 6A-6B show a finger cover system 600 for styling hair and activating hair treatment according to an illustrative embodiments of the invention.

FIG. 7 shows a head covering 700 according to an illustrative embodiment of the invention, a portion of which is shown in cross-section.

FIG. 8 shows a similar embodiment of an external activator, including a dome lamp 800, according to an illustrative embodiment of the invention.

FIG. 9 shows a hair treatment dispenser 900 with a built-in external activator 902, according to an illustrative embodiment of the invention.

FIG. 10 shows a foil treatment according to an illustrative embodiment of the invention.

FIG. 11 shows a mascara wand 1100 according to an illustrative embodiment of the invention.

FIG. 12 shows an eyelash curler 1200 according to an illustrative embodiment of the invention.

FIG. 13 shows a rouge brush 1300 according to an illustrative embodiment of the invention.

FIG. 14 shows a full-body activator 1400 that is configured similar to a tanning bed, according to an illustrative embodiment of the invention.

FIG. 15 shows a lighted tabletop cosmetics mirror according to an illustrative embodiment of the invention.

### DETAILED DESCRIPTION OF CERTAIN EMBODIMENTS OF THE INVENTION

The invention, in various embodiments, provides devices, methods, treatments, and kits for hair treatment and cosmetics applications. The following detailed description of the invention refers to the accompanying drawings. The following detailed description is not intended to limit the invention.

### Hair Treatments

The devices and methods of use of said devices described herein can be used with any hair or cosmetic applications as appropriate. The methods of use of the devices disclosed herein are exemplified with respect to particular formulations and treatment methods, but these exemplary methods should not be construed to limit the uses of the disclosed devices.

In various embodiments the hair treatments initially include monomers, oligomer, or polymers in a solution, and may optionally include a polymerization initiator. Exemplary monomers and polymerization initiators are listed below and in the U.S. and international PCT applications incorporated herein by reference; other suitable monomers and polymerization initiators are known in the art. These monomers, oligomers, polymers, and/or polymerization initiators can be applied using any suitable solvent. A list of exemplary solvents is given below and in the applications incorporated herein by reference; other suitable solvents are known in the art.

After a stylist applies the hair or cosmetic treatment, the stylist uses an external activator, such as a light source or a heat source, to activate the hair or cosmetic treatment. This activation process can include, for example, polymerizing and/or cross-linking monomers, oligomers, or polymers in situ on hair or skin. The hair and cosmetic treatments are formulated such that the resulting polymers impart desirable properties on hair as described herein.

The hair treatment can be provided in a variety of forms, such as in a liquid phase, cream phase, gel phase, wax phase, or mousse phase. In certain embodiments, the stylist or user will choose an appropriate form of the hair treatment in order to suit a particular desired hair style or hair type at hand.

The hair treatment can be provided in a variety of containers for storing and, optionally, dispensing the treatment. By way of example, the hair treatment can be provided in a pump-spray bottle, an aerosol-spray bottle, an aerosol-spray foam dispensing bottle, a squeeze bottle, a squeezable tube, or ajar. The type of container chosen will typically depend on the form of the hair treatment at hand. For example, solution may be in a spray bottle. Gels or creams may be in a squeeze bottle, tube, or jar. In large part, the type of container used will depend on the viscosity of the hair treatment, ease of use, and marketing.

### Methods for Using the Hair Treatments

As mentioned above, exemplary methods of hair treatment include a phase in which a stylist or user applies the hair treatment to hair, and a phase in which the stylist or user activates the hair treatment. Before, during, or after any of these phases, the stylist or user can configure the hair according to a desired hair style.

More particularly, in one exemplary technique the stylist or user first washes the hair to remove dirt or oil. The stylist or user then applies the hair treatment to the hair by, for example, spraying, painting, swabbing, washing, brushing, dipping, soaking, and/or coating the hair with the hair treatment. The method of application will depend on the form of the hair treatment and the container in which it is stored. The stylist or user can simply apply the hair treatment to the hair superficially, or more thoroughly apply the hair treatment by, for example, massaging or rubbing the treatment into the hair. Next, the stylist may optionally configure the hair according to his/her preference. However, in alternative techniques, the stylist or user first configures the hair, then applies the hair treatment, and then, optionally, again configures the hair.

After configuring the hair, the stylist or user activates the hair treatment using an external activator (i.e., a light or heat source) as described herein. However, alternatively, the stylist or user may activate the hair treatment as he/she applies the hair treatment. The stylist or user applies the external activator for a suitable period of time, such as between about 10 seconds and 60 seconds, between about 10 seconds and 40 seconds, between about 10 seconds and 30 seconds, between about 20 seconds and 40 seconds, or between about 20 seconds and 30 seconds. In other techniques, the stylist or user applies the activator for more than 60 seconds, or for less than 10 seconds. As described herein, ranges are used for conciseness only, and any described range is intended to specifically disclose each value within that range. For example a range of from 1 to 5 is intended to include each value within that range, e.g. 1, 1.5, 2, 2.75, etc.

As mentioned above, the hair or cosmetic treatments are formulated such that the polymers formed in situ possess desirable properties or impart desirable properties on the hair or skin. More particularly, after activation, the hair treatments and the generated polymers can impart consistency, durability, definition, pre-selected textures, volume, strength, and/or color to the hair. In various embodiments, the treatment is resistant to flaking or leaving an undesirable residue, even when the hair is subject to abrasive contact. The hair can take on a lustrous or shiny appearance and a natural and smooth feel which is neither sticky nor slimy/slippery to the touch. The treatment can provide strong hold for easy styling by a stylist or the user. In various embodiments, the treatment dries in a relatively short period of time, such as between about 10 seconds and about 10 minutes. In certain embodiments, the treatment dries within 5 minutes. In certain embodiments, the treatment dries within 1 minute.

### External Activators

The stylist or user can activate the hair treatment using a variety of different external activators, which will now be described in connection with the accompanying Figures. As mentioned above, in certain embodiments the external activators are traditional hair care accessories (e.g., brushes, combs, curlers, etc.) that may have, or may be supplemented with, light and/or heat sources. By way of example, the external activators for hair treatment may be hair curling irons, curlers, flat irons, brushes, blow dryers, or combs.

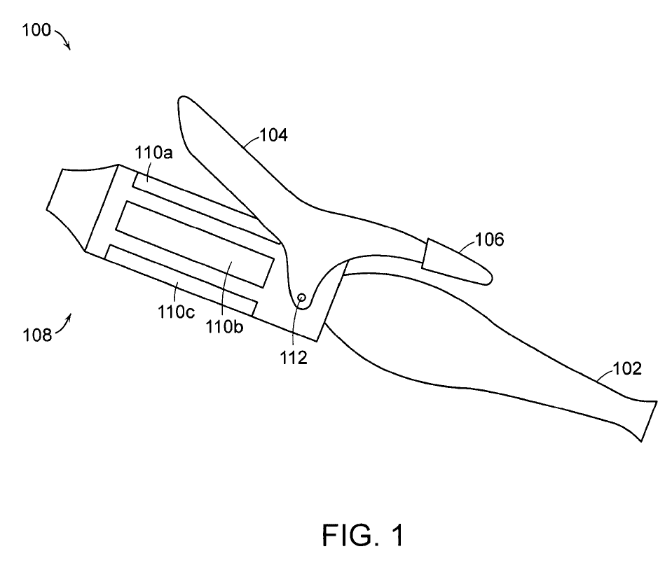

FIG. 1 shows a curling iron 100 according to an illustrative embodiment of the invention. The curling iron 100 includes a handle 102 by which the stylist grasps the curling iron 100, a body 108 around which the stylist wraps hair, and a clasp 104 with which the stylist grips the hair once it has been wrapped around the body 108. Also shown is a lever 106 with which the stylist controllably rotates the clasp 104 about the pivot connector 112 (i.e., a screw or pin). The clasp 104 can rotate from a gripping position, in which the clasp 104 rests on and partially around the body 108, to an open position as depicted in the Figure, in which hair can be disposed between the body 108 and the clasp 104. The stylist uses the curling iron 100 to grasp, curl, uncurl, wave, or otherwise manipulate hair.

In one embodiment, the body 108 and/or the clasp dissipates heat, and the heat activates the hair treatment disposed on the hair. The curling iron 100 can dissipate heat using heating methods known in the art for curling irons. The temperatures needed to initiate the treatment range from about 30° C. to about 120° C. The output temperature of the inventive curling iron is typically in the range of about 50° C. to about 500° C. In certain embodiments, the output temperature is from about 50° C. to about 200° C.

In another embodiment, the iron 100 includes light sources 110a-c which emit light that activates the hair treatment. The light source can be internally located within the body 108, and elements 110a-c can be transparent windows. However, the elements 110a-c can be light sources themselves. In certain embodiments, the light produced is IR, visible, or UV light. In certain embodiments, the UV light produced has a wavelength of from about 200 nm to about 600 nm. In certain embodiments, the UV light has a wavelength of from about 200 nm to about 400 nm. In certain embodiments, the wavelength of the UV light is about 365 nm. In certain embodiments, the intensity of the light is from about 500 μW/cm² to about 10,000 μW/cm². In certain particular embodiments, the intensity of the light is about 7,000 μW/cm².

In various embodiments, elements 110a-c emit ultraviolet light or visible light. By way of example, the light can include components which produce light of wavelengths between about 100 nm and about 750 nm, between about 200 nm and about 600 nm, between about 200 nm and about 350 nm, or between about 400 nm and about 700 nm. However, components with wavelengths in other regions of the electromagnetic spectrum, including those outside of the visible or ultraviolet ranges, may also be used.

Exemplary light sources include blacklights, incandescent light sources (e.g., tungsten lamps), fluorescent light sources (e.g., mercury lamps), xenon arc lamps, deuterium arc lamps, mercury-xenon arc lamps, metal-halide arc lamps, and/or phosphorescent, fluorescent and chemiluminescent light sources. In one exemplary embodiment, the elements 110a-110c are flexible light-emitting-diode (LED) matrices disposed around the body 108 of the inventive curling iron.

In various embodiments, elements 110a-c emit light (or allow transmission of light therethrough in embodiments where the elements 110a-c serve as windows for an internal light source) with an intensity of between about 500 and about 10,000 micro-watts per square centimeter, or about 7000 micro-watts per square centimeter. In other embodiments the core of the curling iron does not emit heat and/or light but rather the clasp emits the light and/or heat. In other embodiments, the iron itself does not emit heat or light; instead, the hair is wrapped around the iron as usual and a sleeve or cover which emits light and/or heat is placed over or around the iron.

In an exemplary usage technique, the operator grasps the iron 100, opens the clasp 104 using lever 106, disposes a target tress of hair between the clasp 104 and the body 108, releases the lever 106 to close the clasp 104, and manipulates the tress of hair as desired (i.e., to curl the hair). The stylist or user can manipulate the tress of hair with the heat or light emanating from the body 108, or can first manipulate the tress of hair, and then turn on the heat or light elements 110a-c. As mentioned above, the stylist or user applies the heat or light to the tress for between about 10 seconds and about 60 seconds. In certain embodiments, the stylist or user applies heat or light to the tress of hair for between about 10 seconds and 40 seconds. The stylist or user then releases the tress of hair using lever 106, and optionally repeats the procedure for another portion of hair.

In order to generate the light or heat, the iron 100 may include or couple to a power source (not shown) such as a battery or an electrical connection to an electrical wall outlet.

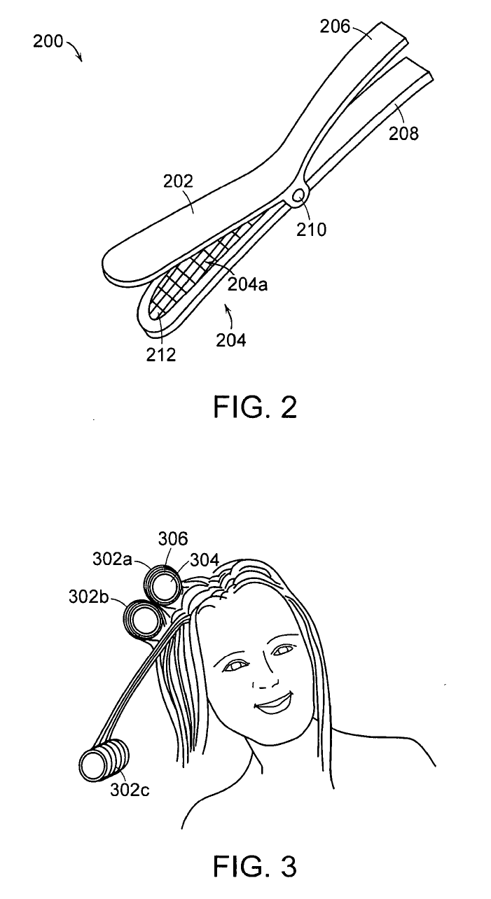

FIG. 2 shows a flat iron 200 according to an illustrative embodiment of the invention. The flat iron is configured and operates similarly to the curling iron 100, in that it includes a handle 208, a body 204, a clasp 202, a lever 206, and a pivot 210. However, flat iron 200 differs from curling iron 200 in that the body 204 includes a planar inner surface 204a and the clasp 202 includes a planar inner surface (not shown) suitable for grasping and flattening/straightening hair. As with body and clasp 108 and 104 of iron 100, body 204 and clasp 206 of iron 200 can include a light and/or heat source for activating the hair treatment. The heat can be generated using heating methods known in the art for flat irons. Depicted in the Figure is a light array 212 that is integral to the body 204 and aligns with the inner surface 204a. The light source can be internal to the body 204, or can be disposed on the inner surface 204a as depicted. The light source can be any of those mentioned above for the curling iron 100. In certain embodiments, the flat iron comprises a cover or sleeve which emits radiation (e.g., heat and/or light) instead of or in addition to the body of the iron. The flat iron is used similarly to the curling iron described above.

FIG. 3 shows hair curlers 302a-c according to an illustrative embodiment of the invention. As shown with respect to curler 302c, tresses of hair wrap around respective hair curlers 302a-c, which can then be set in place, as shown with respect to curlers 302a-b. The curlers 302a-c can be used to curl or wave hair. The curlers 302a-c can include heat or light sources for activating the hair treatment. More particularly, the curler 302a can include a light source or heat source, such as any of the light sources or heat sources described above, within its core 304. Alternatively, or additionally, the curler 302a can include a light source disposed around the outer surface 306 of the curler 302a. In other embodiments, the curler can comprise bristles which emit radiation, e.g., as described herein for brush-like devices. In other embodiments, the curler can comprise a cover or sleeve which emits radiation (e.g., heat and/or light) instead of or in addition to the core of the curler. In use, the stylist wraps the tresses of hair around the curlers 302a-c, sets the curlers 302a-c, and then initiates the activation of the hair treatment by, for example, powering on the curlers 302a-c (power sources not shown) so they emit light and/or heat, and/or by powering on the cover or sleeve (placed over the hair wrapped around the curler core) to emit heat and/or light.

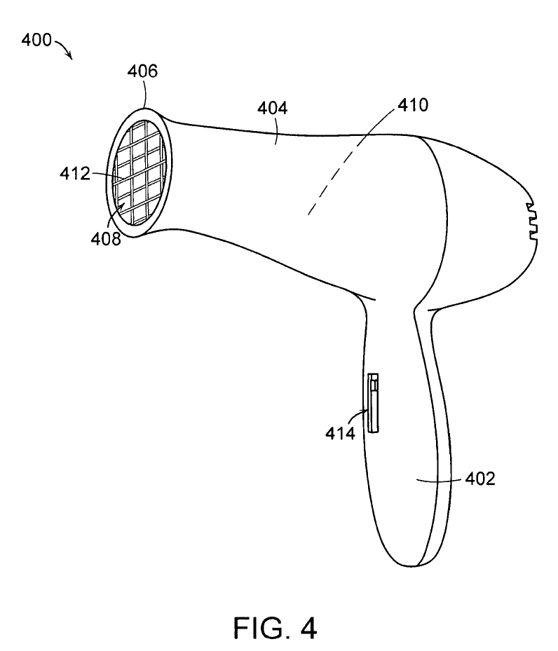

FIG. 4 illustrates an external activator 400 configured like a blow-dryer, according to an illustrative embodiment of the invention. While FIGS. 1-3 illustrate devices with which the stylist or user can both style/shape hair and activate the hair treatment, FIG. 4 illustrates a device 400 that is used primarily to activate the hair treatment. The activator 400 includes a handle 402 with which the stylist or user grasps the activator 400 and a body 404. In certain embodiments, the activator 400 functions as a standard hair blow dryer, by generating and blowing hot air through the surface 408. This hot air thereby provides the heat to activate the hair treatment.

In the embodiment just described, the blow dryer 400 heats the hair and the hair treatment by convection. However, in the depicted embodiment, the activator 400 heats by radiation, and includes a radiator (not shown) and a radiator grill 412. Heating by radiation may be beneficial compared to heating by convection for this purpose at least in part because heating by convection may blow air towards hair that has already been configured by the stylist, thereby unconfiguring or “messing-up” the hair.

In addition or as an alternative to heat, the activator 400 may emit light to activate the hair treatment. In the depicted embodiment, the activator 400 also includes a tube-light 406 disposed around the periphery of the surface 408. The tube-light 406 can comprise any of the light sources described above. In certain embodiments, the light produced is IR, visible, or UV light. In certain embodiments, the UV light produced has a wavelength of from about 200 nm to about 600 nm. In certain embodiments, the UV light has a wavelength of from about 200 nm to about 400 nm. In certain embodiments, the wavelength of the UV light is about 365 nm. In certain embodiments, the intensity of the light is from about 500 μW/cm² to about 10,000 μW/cm². In certain particular embodiments, the intensity of the light is about 7,000 μW/cm². In an alternative embodiment, the activator 400 does not include a heat source, but includes a light source disposed in the core 410 of the body 404. The light can shine through the surface 408.

In use, when the stylist or user is ready to activate the hair treatment, he/she points the surface 408 of the activator 400 to a target portion of hair, and emits light and/or heat towards the hair by, for example, powering the device on with a switch 414. The intensity, temperature, and wavelength of the light and/or heat may be adjusted by various controls on the blow dryer (not shown).

In certain embodiments, even if heat (convection or radiation) or non-visible portions of the electromagnetic spectrum are used to activate the hair treatment, the activator still includes a visible light source, such as tube light 406 or a light source located in the core 410. This can be beneficial so that the stylist has a visible guide as to which region of the hair he/she is activating during use. The use of visible light as a visual cue to the stylist for the region of hair being activated can be used in any suitable device or embodiment described herein.

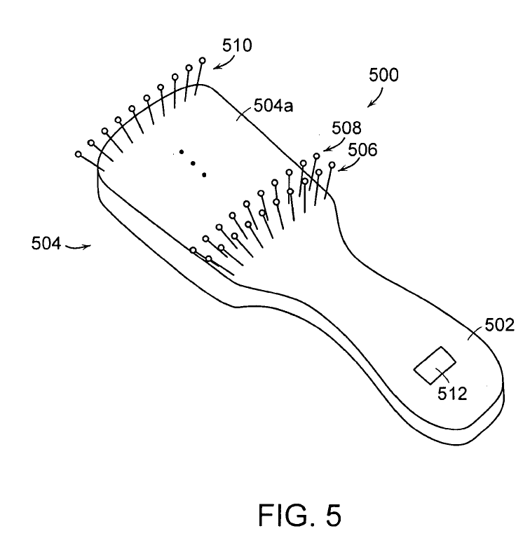

Other devices (as with FIGS. 1-3) that can optionally be used to simultaneously style hair and activate the hair treatment include brushes and combs. FIG. 5 shows a hair brush 500 according to an illustrative embodiment of the invention. The hair brush 500 includes a handle 502 and a body 504. The body 504 includes a plurality of rows of bristles, although only three exemplary rows 506, 508, and 510 are shown. In certain embodiments, the bristles 506, 508, and 510 emit light. By way of example, the bristles 506, 508, and 510 can comprise luminescent filaments, light strips, or thin tubes that include light sources therein. Exemplary light strips include LED light strips. In embodiments where a portion of the visible light spectrum is used, the rows 506, 508, and 510 may be of the same color, may be of different color, and may periodically change color for visual appeal.

The device 500 may also dissipate heat from body 500. In certain embodiments, the surface 504a of the body 504 dissipates heat. The bristles 506, 508, and 510 may comprise thermally conductive materials, and draw heat towards the hair treatment to activate the hair treatment. In certain embodiments, the handle 502 is thermally insulated from the body 504 so the handle remains at a comfortable temperature as the stylist or user uses the brush 500 to treat the hair. In use, the stylist runs the bristles 506, 508, and 510 through the portion of the hair on which the stylist wishes to activate the hair treatment.

The above-described features may also apply to a comb. More particularly, the variations described with respect to the bristles 506, 508, and 510 may apply to the teeth of a comb.

In addition to traditional hair care devices that either emit or are supplemented to include components that emit light and/or heat, such as those mentioned above, the external activators for use with the hair treatment can include other devices, such as devices that a person may wear.

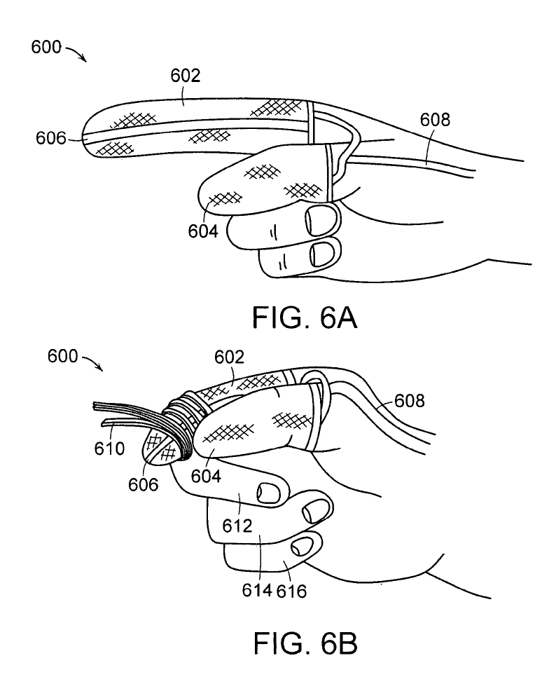

FIGS. 6A-6B show a finger cover system 600 for styling hair and activating hair treatment according to an illustrative embodiments of the invention. In general, a stylist wears the system 600 on his/her forefinger and thumb (FIG. 6A), and styles hair (FIG. 6B). The system 600 includes light or heat sources for activating the hair treatment in order to facilitate the styling. In particular, the system 600 includes a forefinger cover 602 and a thumb cover 604. The forefinger cover 602 includes an activator strip 606 for activating the hair treatment. In certain embodiments, the activator strip is a light source, such as a light filament, and can comprise any of the light sources described above. In other embodiments, the activator strip 606 is a thermally conductive heating element that applies heat to the hair treatment to activate the hair treatment. The activator strip 606 may be disposed (i.e., bonded with an adhesive) on the forefinger cover 602, and the forefinger cover 602 may comprise a thermally insulating material too so that the inner surface of the forefinger cover 602 stays at a comfortable temperature for the stylist. Although the activator strip 606 is depicted as a long and thin strip, in other embodiments the activator strip 606 covers more of the forefinger cover 602 and may even cover the entire forefinger cover 602. In other embodiments, the activator strip 606 is not present, and the forefinger cover 602 itself emits light and/or heat.

Also shown is a thumb cover 604. A surface (not shown) of the thumb cover 604 also includes an activator strip like the activator strip 606, so that the stylist can pinch a tress of hair and have both the activator strip 606 of the forefinger cover 602 and the activator strip (not shown) of the thumb cover 604 emit light and/or heat on the hair. The operator can use his/her forefinger and/or thumb to style the hair as desired, such as to curl, straighten, or otherwise manipulate and configure the hair. By way of example, FIG. 6B shows the stylist curling a tress 610 of hair.

The activator strips, including activator strip 606, may require power in order to emit light and/or heat. A power cord 608 is depicted, which electrically couples to a wall outlet or another power source, such as a battery pack.

The system 600 only includes covers for the stylist's forefinger and thumb, which may be beneficial at least in part so that the stylist's other remaining three fingers 612, 614, and 616 are available to touch the hair and provide tactile feedback to the stylist. However, in other embodiments, 1, 3, 4, or 5 of the fingers have covers. In one embodiment, a glove is provided that includes activator strips such as strip 606 on 1, 2, 3, 4, or 5 of the finger covers of the glove. Such a glove may include a palm region that covers the palm of the stylist or user. The palm region may also emit light and/or heat. The stylist or user may use an inventive system 600 on one or both hands.

While in one exemplary technique the stylist or user applies the hair treatment to the tress 610 first and then styles the tress of hair, in others the stylist applies the hair treatment using the system 600, such as by dipping the finger cover 602 into a hair treatment gel or cream and then disposing the hair treatment on the hair from the system 600. In certain techniques, the stylist powers on the activator strip for emitting light and/or heat during application of the hair treatment to the hair.

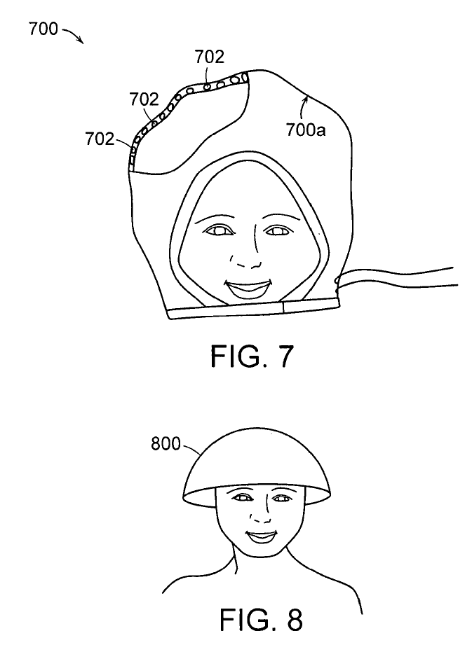

FIG. 7 shows a head covering 700 according to an illustrative embodiment of the invention, a portion of which is shown in cross-section. The head covering 700 emits light and/or heat on the hair of the person wearing the head covering 700 to activate the hair treatment in his/her hair. More particularly, after the stylist applies the hair treatment and configures the hair, the head covering 700 is then placed on the head of the person being styled. The depicted head covering 700 includes a plurality of thermally conductive elements 702 (i.e., thermally conductive wires or filaments) that distribute heat throughout the head covering 700 so that a substantially uniform field of heat is generated around the hair. Alternatively, the head covering 700 can include light emitters on an inner surface 700a of the head covering 700, such as any of the light sources described above.

FIG. 8 shows another external activator that is disposed around a person's head according to an illustrative embodiment of the invention. This activator is a dome lamp 800, which can be a heat lamp and/or a light lamp. As shown, the stylist places the dome lamp 800 over the hair being treated. The stylist then powers the dome lamp 800 on to emit heat and/or light. The dome lamp 800 can include any of the heat and/or light sources described above.

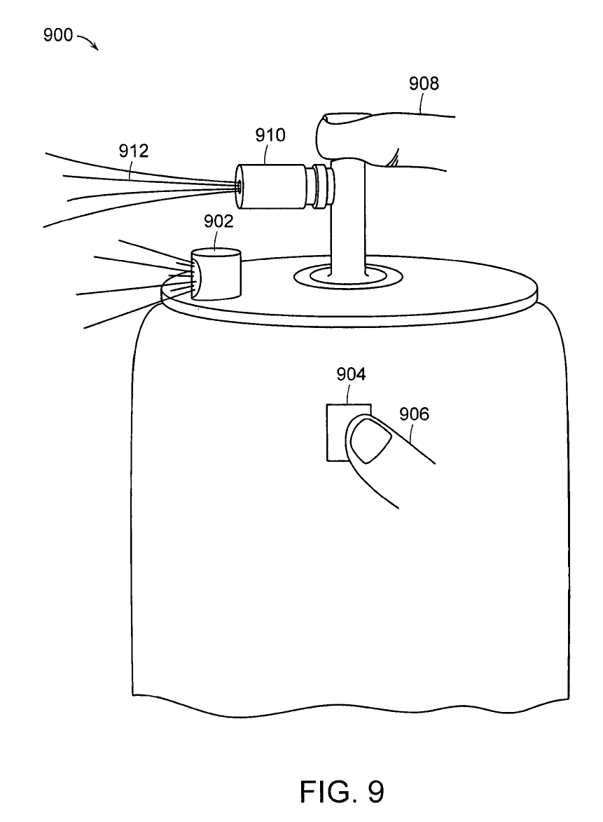

FIG. 9 shows a hair treatment dispenser 900 with a built-in external activator 902, according to an illustrative embodiment of the invention. Since the external activator 902 is included integrally within a hair treatment dispenser 900, the stylist or user only needs to use one device to both apply the hair treatment and activate the hair treatment. More particularly, the dispenser 900 is an aerosol spray can. The stylist pushes down on the nozzle 910 using forefinger 908, which releases a stream 912 of hair treatment. Simultaneously, or afterwards, the stylist uses a thumb 906 to push the button 904, which activates the light 902. By disposing the button 904 in a location accessible to the thumb 906, the stylist can controllably operate both the hair treatment nozzle 910 and the light 902 simultaneously. As mentioned above, the light 902 may be visible or non-visible light. In other embodiments, element 902 is a heat source rather than a light source. However, even in embodiments where a heat source or a non-visible light source is used, there may be a visible light component as well to serve as a guide to the stylist or user as to where the activation of the hair treatment is taking place. In certain embodiments, the light source 902 is an inexpensive or commercially available light source, such as an LED, and thus the dispenser 900 is a one-time use disposable dispenser. In another example 902 is a solution of a polymerization initiator. This is beneficial at least in part so that consumers only need to purchase one economical product (device 900) in order to make use of the hair treatments described herein and in the patent applications referenced herein. Similar configurations can be used for any of the non-aerosol containers and/or dispensers described herein.

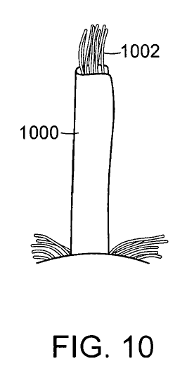

The hair treatments can treat individual tresses of hair, and, as mentioned above, color hair. One exemplary application is highlighting (or lowlighting) hair. As an example, FIG. 10 shows a foil treatment according to an illustrative embodiment of the invention. In use, the stylist or user treats the tress 1002 of hair with the hair treatment, which includes coloring, highlighting, and/or lowlighting agents. The stylist then covers the tress 1002 of hair with a sheet of foil 1000. The foil 1000 is thermally conductive, and is heated by a heat source (not shown) such as a heat source directly coupled to the foil 1000, or by any of the other devices described herein, such as the dome lamp 800, to activate the hair treatment. The foil 1000 can comprise any thermally conductive material that is sufficiently shapeable to allow the stylist to encapsulate the tress 1002 of hair. In other embodiments, instead of a sheet of foil 1000, the stylist uses a tube, such as an expandable and collapsible tube. The stylist expands the tube, guides the tress of hair through the tube, and then collapses the tube to encapsulate the tress of hair. The tubes of foils 1000 may also be provided with light sources in addition or as an alternative to the heat source.

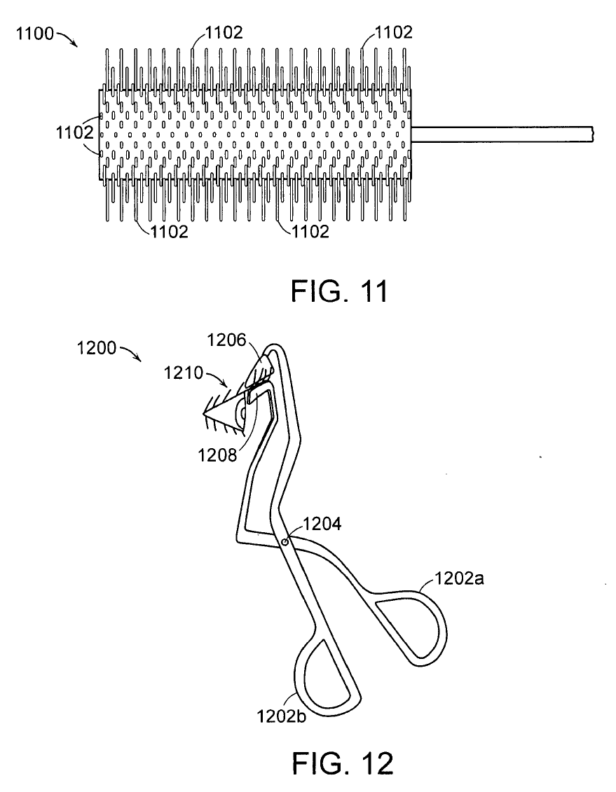

The systems, devices, methods, and kits described herein are not limited to hair on top of a human head. They can also be applied to animals, such as pets, as well as other human hair, such as facial hair (mustaches and beards), eyebrows, or eyelashes, and wigs. By way of example, many women curl their eyelashes, but when using existing curling techniques, the eyelashes do not adequately retain their curled configuration. FIG. 11 shows a mascara wand 1100 according to an illustrative embodiment of the invention, and FIG. 12 shows an eyelash curler 1200 according to an illustrative embodiment of the invention. Small combs and brushes suitable for use with the inventive hair treatment in styling a beard or mustache are also provided by the invention.

More particularly, in use, the stylist or user dips the mascara want 1100 into the hair treatment. The stylist or user then applies the hair treatment to the eyelashes as mascara is typically applied. The mascara wand 1100 includes bristles, of which certain exemplary bristles 1102 are depicted. The bristles may take on any of the alternative embodiments of the bristles 506, 508, and 510 discussed in connection with FIG. 5 (i.e., they may emit light and/or heat) in order to activate the treatment. By using the mascara wand 1100 on the inner surfaces of eyelashes (i.e., the lower surface of the top eyelash and the upper surface of the bottom eyelash), the stylist may thus curl the eyelashes, and due to the activation of the hair treatment, the eyelashes will remain in the curled configuration. Alternatively, the hair treatment may be applied to the outer surfaces of the eyelashes for that ‘heavy-lidded’ look. In other embodiments, the stylist or user applies the hair treatment using a conventional mascara wand, and then activates the hair treatment using any of the external activators described herein.

Optionally, in another embodiment, the stylist or user may use the eyelash curler 1200 after applying the hair treatment to the eyelash 1210. The eyelash curler 1200 includes handle portions 1202a-b, a pivot 1204, an inner eyelash-contacting surface 1206, and an outer eyelash-contacting surface 1208. In use, the stylist positions the device 1200 with respect to the eyelash 1210 as depicted in FIG. 12, with the eyelash 1210 disposed between the inner eyelash-contacting surface 1206 and the outer eyelash-contacting surface 1208. The operator then squeezes together the handle portions 1202a-b to draw the outer eyelash-contacting surface 1208 flush against and interfitting around the inner eyelash-contacting surface 1206, with the eyelash 1210 still disposed therebetween and taking on a curled-up configuration. In certain embodiments, the eyelash curler 1200 thermally couples to a heat source (not shown) so that the surfaces 1206 and 1208 heat to activate the eyelash 1210. As with other devices described herein, the handles 1202a-b will be thermally isolated for stylist comfort.

### Cosmetics

A stylist or user can use similar techniques and devices for cosmetic treatments. As mentioned above, the methods for using the cosmetics generally include two phases: in a first phase, a stylist applies the cosmetic to skin; and in a second phase, the stylist activates the cosmetics by, for example, applying light or heat to the or cosmetic.

The principle of operation for the cosmetics is similar to that of the hair treatments. More particularly, the cosmetics, in various embodiments, initially (i.e., before being applied to skin) include monomers, oligomers, or polymers in a solution. The solution may also include an initiator, such as a polymerization initiator, that, upon exposure to an external activator (e.g. light or heat), initiates the activation of the cosmetics. After a stylist or user applies the cosmetics, the stylist uses an external activator, such as a light source or a heat source, to activate the cosmetics. This activation process can include, for example, polymerization and/or cross-linking the monomers, oligomers, or polymers to generate polymers, optionally cross-linked, in situ. The cosmetics are formulated such that the formed polymers impart desirable properties on skin. Additionally, it has been discovered that this process is particularly effective because in situ activation of the cosmetics further enhances these desirable properties.

The cosmetics can be provided in a variety of forms and in a variety of containers and dispensers. More particularly, the cosmetics can be provided in a liquid phase, a cream or lotion phase, a gel phase, or a powder phase. The cosmetics can be standalone cosmetic products, as will be described below, or can be integrated with other products such as soaps, lotions, creams, scrubs, etc. The cosmetics can be provided in any of the containers discussed above with respect to hair treatments, and can also be provided in other types of make-up kits such as compacts.

The cosmetics can comprise various types of makeup. By way of example, the cosmetics can comprise lipstick, lip gloss, foundation, powder, rouge (alternately blush or blusher), mascara (as described above in connection with hair treatments), eyeliner, eyeshadow, fingernail polish, toenail polish, concealer, and wrinkle treatment.

In use, the stylist can apply these cosmetics products to skin or nails using techniques known in the art and then use any of the external activators described above to activate the cosmetics. By way of example, in one technique the stylist or user points the activator 400 of FIG. 4 towards the skin, powers on the activator 400, and the activator 400 then emits light and/or heat to activate the cosmetic treatment. In another technique, the stylist or user uses the system 600 and, for example, runs the forefinger cover 602 over the skin on which the stylist or user applied the cosmetics. The stylist or user may also apply the cosmetics to the skin using the forefinger cover 602, as was described in connection with hair treatments. The stylist or user may cover the entire head of the person being styled with a head covering 700 (FIG. 7) or a dome 800 (FIG. 8). In certain embodiments, the head covering 700 does not include the depicted opening for the front of the face, and instead covers the whole head including the front of the face, or only covers the front of the face. In certain embodiments, the head covering 700 may include the neck and/or shoulders. Such coverings may also be designed for the extremities (e.g., legs, arms), hands, feet, torso, chest, or whole body.

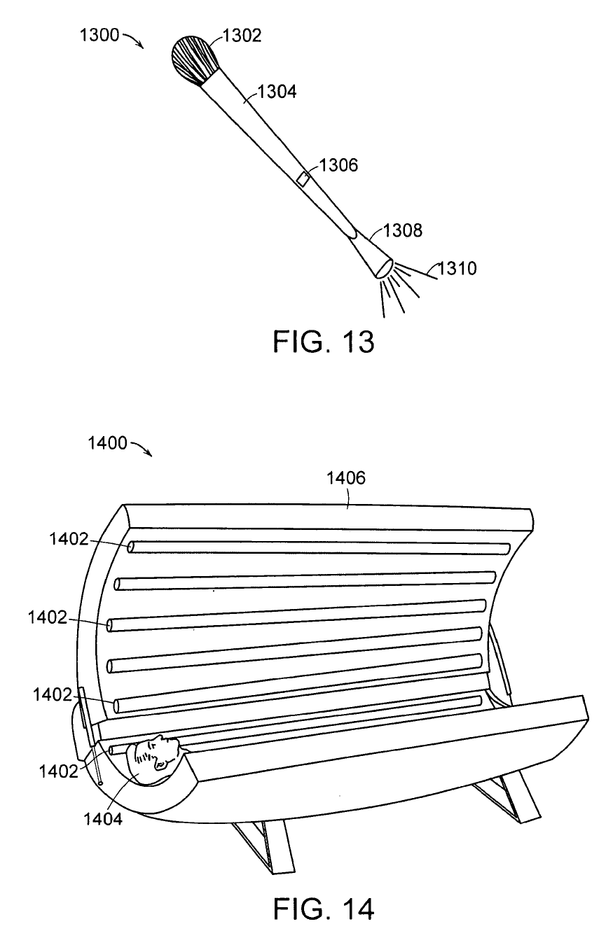

As with hair treatment methods described above, certain cosmetic treatments include devices that can be used as both applicators and activators. FIG. 13 shows a rouge brush 1300 according to an illustrative embodiment of the invention. The rouge brush 1300 includes a body 1304 and bristles 1302 for applying rouge to a face using application methods as are known in the art, but also includes an external activator 1308 that emits light or heat 1310 when a stylist or user presses the button 1306. Thus, the stylist or user can apply cosmetics using the bristles 1302, and activate the cosmetics using the light 1308 on the opposite end of the body 1304. This is beneficial at least in part so that the stylist only needs one device to both apply the cosmetics and to activate the cosmetics. Similar variations can be applied to other cosmetics applications. For example, lip stick applicants can have an external activator coupled to a back end of the lip stick applicator.

Alternatively, or additionally, the bristles 1302 may be heated bristles. In use, the stylist or user turns on the heat from the bristles 1302 using a button similar to the button 1306, and this activates the cosmetics being applied with the brush.

The stylist or user may also apply cosmetics to portions of the body beyond the face or head. In fact, one exemplary technique offers a full body treatment. More particularly, FIG. 14 shows a full-body activator 1400 that is configured similar to a tanning bed. A full body activator configured similarly to a tanning booth may also be used. In use, the stylist applies a cosmetic, such as an anti-wrinkle cosmetic or a moisturizing cosmetic, all over the body of a person 1404. Next, the person 1404 lies in the full body activator 1400. The full-body activator 1400 includes a plurality of bulbs 1402 which emit light or heat to activate the cosmetics applied to person 1404. The bulbs 1402 may comprise any of the light or heat sources described herein. The cover 1406 of the full-body activator 1400 is closed, and the bulbs 1402 are turned on. The person 1404 remains in the full-body activator for a suitable period of time, such as between 20 seconds and 1 minutes, until the cosmetics activate. In certain embodiments, the light produced is IR, visible, or UV light. In certain embodiments, the UV light use has a wavelength of from about 200 nm to about 600 nm. In certain embodiments, the UV light has a wavelength of from about 200 nm to about 400 nm. In certain embodiments, the wavelength of the UV light is about 365 nm. In certain embodiments, the intensity of the light is from about 500 μW/cm² to about 10,000 μW/cm². In certain particular embodiments, the intensity of the light is about 7,000 μW/cm².

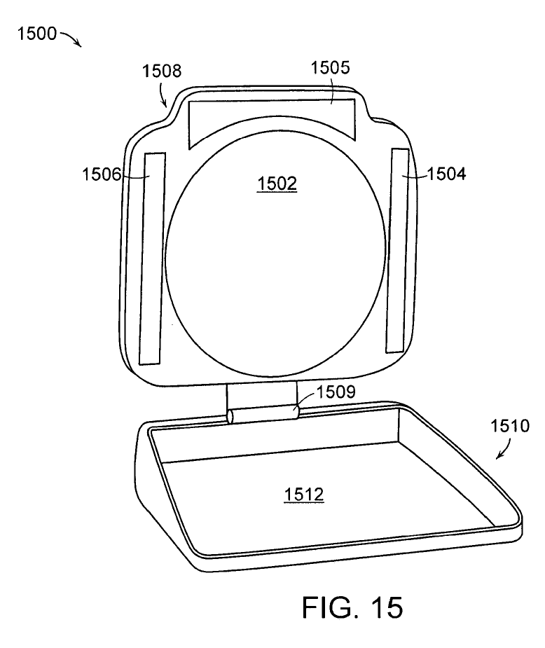

FIG. 15 shows a lighted tabletop cosmetics mirror (“compact” mirror) according to an illustrative embodiment of the invention. The device 1500 includes a base 1510 and a pivotable top 1508 that pivots about the hinge 1509 so a user can vary the device 1500 from the depicted open position, to a closed position wherein the top 1508 lies directly on top of or within the base 1510.

The pivotable top includes a mirror 1502 and light sources 1504-1506. The depicted light sources 1504-1506 can be any of the light sources described herein, and they emit light to activate facial cosmetics and/or hair treatments of a person using the device 1500. The depicted light sources 1504-1506 surround the mirror 1502, and may surround a portion or all of the mirror 1502. In other embodiments, light sources are disposed directly on the mirror 1502. Additionally or alternatively, heat sources, such as any of the heat sources described herein, may be disposed around or on the mirror 1502. Additionally or alternatively, light and/or heat source may be disposed on or in the base 1510, which may also be used as a storage container for cosmetics, cosmetics accessories, hair treatments, or hair treatment accessories.

In addition to cosmetics mirrors such as the depicted device 1500, heat and/or light sources can be disposed on or near handheld mirrors, full length mirrors, or any other type of mirror.

### Exemplary Embodiments for Hair and Cosmetic Treatments

It is to be understood that any of the hair or cosmetic treatments may be functional (i.e., impart desired properties onto hair and/or skin) even when the hair treatments and cosmetics are not activated by, for example, light or heat. Thus, in certain embodiments, activating the cosmetics merely enhances one or more of the desired properties. However, in other embodiments, the activation imparts properties that were not present in the cosmetics before activation. For example, in certain embodiments, the activation step creates polymers and/or cross-links polymers.

Any of the devices described herein can be powered by an appropriate energy source, such as a battery, fuel cell, portable (i.e., micro) fuel cell, electrical connection to a wall outlet, solar cell, or the like.

As described above, the hair and cosmetic treatments initially include monomers, oligomers, or polymers that are activated (i.e., polymerized or cross-linked) in situ. The polymerizable monomer comprises a functional group suitable for polymerization. Any functional group that can be polymerized using a free radical or ionic polymerization reaction can be used. In certain embodiments, the monomers include a functional group with at least one degree of unsaturation. For example, the monomer includes a double bond or triple bond. Exemplary functional groups suitable for polymerization include alkenes, alkynes, carbonyls, imines, thiocarbonyls, acrylates, methacrylates, acrylates, crotonates, styrenes, nitriles, cyano, vinyl, styrene, crotonate, cinnamate, dienes, trienes, eneynes, maleimides, etc. In certain particular embodiments, the monomers comprise a vinyl group. In certain particular embodiments, the monomers comprise an acrylate functional group. In certain particular embodiments, the monomers comprise a methacrylate functional group. In certain particular embodiments, the monomers comprise a diene moiety. In certain embodiments, the monomers comprise a conjugated diene moiety. In certain embodiments, the monomers comprise a maleimide moiety. Other reactive functional groups may also be used including epoxides and halogen-containing compounds.

Exemplary monomers useful in accordance with the present invention include trimethylolpropane trimethacrylate; 1,3-bis(3-methacryloyloxypropyl)-1,1,3,3-tetramethyldisiloxane; 1,3-butanediol dimethacrylate; 1,4-butanediol dimethacrylate; 1,6-hexanediol dimethacrylate; bisphenol A dimethacrylate; bisphenol A ethoxylate dimethacrylate; bisphenol A glycerolate dimethacrylate; di(ethylene glycol) dimethacrylate; diurethane dimethacrylate, mixture of isomers; ethylene glycol dimethacrylate; glycerol dimethacrylate, mixture of isomers; neopentyl glycol dimethacrylate; poly(ethylene glycol) dimethacrylate; poly(lauryl methacrylate-co-ethylene glycol dimethacrylate); poly(methyl methacrylate-co-ethylene glycol dimethacrylate); poly(propylene glycol) dimethacrylate; tetraethylene glycol dimethacrylate; triethylene glycol dimethacrylate; 1,1,1,3,3,3-hexafluoroisopropyl methacrylate; 2-(9H-carbazol-9-yl)ethyl acrylate; 2-(diethylamino)ethyl methacrylate; 2-(dimethylamino)ethyl methacrylate; 2-(methacryloyloxy)ethyl acetoacetate; 2-(methylthio)ethyl methacrylate; 2-(tert-butylamino)ethyl methacrylate; 2-(trimethylsilyloxy)ethyl methacrylate; 2,2,2-trifluoroethyl methacrylate; 2,2,3,3,3-pentafluoropropyl methacrylate; 2,2,3,3,4,4,4-heptafluorobutyl methacrylate; 2,2,3,3,4,4,5,5-octafluoropentyl methacrylate; 2,2,3,4,4,4-hexafluorobutyl methacrylate; 2-[3-(2H-benzotriazol-2-yl)-4-hydroxyphenyl]ethyl methacrylate; 2-aminoethyl methacrylate hydrochloride; 2-butoxyethyl methacrylate; 2-ethoxyethyl methacrylate; 2-ethylhexyl methacrylate; 2-hydroxyethyl methacrylate; 2-methyl-2-nitropropyl methacrylate; 2-naphthyl methacrylate; 3-(acryloyloxy)-2-hydroxypropyl methacrylate; 3-(diethoxymethylsilyl)propyl methacrylate; 3-(dimethylchlorosilyl)propyl methacrylate; 3-(trichlorosilyl)propyl methacrylate; 3-(dimethylchlorosilyl)propyl methacrylate; 3-(trichlorosilyl)propyl methacrylate; 3-(trimethoxysilyl)propyl methacrylate; 3,3,4,4,5,5,6,6,6-nonafluorohexyl methacrylate; 3,3,4,4,5,5,6,6,7,7,8,8,8-tridecafluorooctyl methacrylate; 3,3,4,4,5,5,6,6,7,7,8,8,9,10,10,10-hexadecafluoro-9-trifluoromethyl)decyl methacrylate; 3,3,4,4,5,5,6,6,7,7,8,8,9,9,10,10,10-heptadecafluorodecyl methacrylate; 3,3,4,4,5,5,6,6,7,7,8,8,9,9,10,10,11,11,12,12,12-heneicosafluorododecyl methacrylate; 3,3,4,4,5,5,6,6,7,7,8,8,9,9,10,10,11,12,12,12-eicosafluoro-11-(trifluoromethyl)dodecyl methacrylate; 3,3,4,4,5,5,6,6,7,8,8,8-dodecafluoro-7-(trifluoromethyl)octyl methacrylate; 3,3,4,4,5,6,6,6-octafluoro-5-(trifluoromethyl)hexyl methacrylate; 3,3,5-trimethylcyclohexyl methacrylate, mixture of isomers; 3-[(3,5,7,9,11,13,15-heptacyclopentylpentacyclo[9.5.1.1^{3,9}.1^{5,15}.1^{7,13}]octasiloxan-1-yloxy)dimethylsilyl]propyl methacrylate; 3-[tris(trimethylsiloxy)silyl]propyl methacrylate; 3-chloro-2-hydroxypropyl methacrylate; 3-sulfopropyl methacrylate; 4,4,5,5,6,6,7,7,8,8,9,9,10,11,11,11-hexadecafluoro-2-hydroxy-10-(trifluoromethyl)undecyl methacrylate; 4,4,5,5,6,6,7,7,8,9,9,9-dodecafluoro-2-hydroxy-8-(trifluoromethyl)nonyl methacrylate; 4,4,5,5,6,7,7,7-octafluoro-2-hydroxy-6-(trifluoromethyl)heptyl methacrylate; 6-[4-(4-cyanophenyl)phenoxy]hexyl methacrylate; 9-anthracenylmethyl methacrylate; 9H-carbazole-9-ethylmethacrylate; allyl methacrylate; benzyl methacrylate; butyl methacrylate; cyclohexyl methacrylate; decyl methacrylate; di(ethylene glycol) ethyl ether methacrylate; di(ethylene glycol) methyl ether methacrylate; di(propylene glycol) allyl ether methacrylate, mixture of isomers; Disperse Red 1 methacrylate; Disperse Red 13 methacrylate; Disperse yellow 7 methacrylate; ethyl methacrylate; ethylene glycol dicyclopentenyl ether methacrylate; ethylene glycol methyl ether methacrylate; ethylene glycol phenyl ether methacrylate; furfuryl methacrylate; glycidyl methacrylate; glycol methacrylate; glycosyloxyethyl methacrylate; hexyl methacrylate; hydroxybutyl methacrylate, mixture of isomers; hydroxypropyl methacrylate; isobornyl methacrylate; isobutyl methacrylate; isodecyl methacrylate; lauryl methacrylate; methyl methacrylate; stearyl methacrylate; tert-butyl methacrylate; tetrahydrofurfuryl methacrylate; tridecyl methacrylate; trimethylsilyl methacrylate; vinyl methacrylate; glycerol propoxylate (1PO/OH) triacrylate; pentaerythritol triacrylate; trimethylolpropane ethoxylate triacrylate; trimethylolpropane propoxylate triacrylate; trimethylolpropane triacrylate; di(trimethylolpropane) tetraacrylate; pentaerythritol tetraacrylate; dipentaerythritol pentaacrylate; ethoxylated pentaerythritol tetraacrylate; low viscosity dipentaerythritol pentaacrylate; pentaacrylate ester; pentaerythritol tetraacrylate; trimethylolpropane triacrylate; ethoxylated trimethylolpropane triacrylate; propoxylated glycerol triacrylate; pentaerythritol triacrylate; propoxylated glyceryl triacrylate; propoxylated trimethylolpropane triacrylate; trimethylolpropane trimethacrylate; tris(2-hydroxy ethyl) isocyanurate triacrylate; tris(2-hydroxy ethyl) isocyanurate triacrylate; polybutadiene diacrylate; and polybutadiene dimethacrylate. In certain particular embodiments, the monomer is ethyl acrylate; vinyl acrylate; 1,3-butanediol diacrylate; dipentaerythritol pentaacrylate; tridecyl methacrylate; styrene; and 3,4-epoxycyclohexylmethyl 3′,4′-epoxycyclohexane carboxylate. In certain embodiments, the monomer is a polybutadiene di(meth)acrylate oligomer. In certain embodiments, the monomer is tricyclodecane dimethanol diacrylate. In certain embodiments, the monomer is tricyclodecane dimethanol dimethacrylate.

In certain embodiments, a fluorinated monomer is polymerized on hair or skin based on the inventive hair or cosmetic treatment system. The fluorinated monomer comprises a functional group suitable for polymerization and at least one fluorine atom. Any functional group that can be polymerized using a free radical or ionic polymerization reaction can be used. Certain such functional groups are described. Typically, the functional group includes a degree of unsaturation (e.g., a double bond or triple bond). Exemplary functional groups suitable for polymerization include alkenes, alkynes, carbonyls, imines, thiocarbonyls, acrylates, methacrylates, acrylates, crotonates, styrenes, nitriles, cyano, vinyl, styrene, crotonate, cinnamate, dienes, trienes, eneynes, maleimides, etc.

The fluorinated monomer may range from including one fluorine atom to being perfluorinated. In certain embodiments, a functional group of the monomer is perfluorinated such as, for example, an alkyl, alkenyl, alkynyl, acyl, aryl, heteroaryl, heterocyclic, or carbocyclic moiety. In certain embodiments, the fluorinated monomer includes at least 1, 2, 3, 4, 5, 6, 7, 8, 9, 10, 11, 12, 13, 14, 15, 16, 17, 18, 19, or 20 fluorine atoms. In other embodiments, the fluorinated monomer contains at least 10, at least 15, at least 20, at least 25, at least 30, or at least 40 fluorine atoms. As would be appreciated by one of skill in this art, the larger the monomer the more fluorine atoms the monomer is likely to have. Furthermore, the monomer should include enough fluorine atoms so that the resulting polymer imparts the desired characteristics when polymerized on skin (e.g., appearance, robustness, feel, styling effect).

In certain embodiments, at least 10%, 20%, 30%, 40%, 50%, 60%, 70%, 80%, 90%, 95%, or 99% of the total number of hydrogen and fluorine atoms are fluorine atoms in the fluorinated monomer. In certain embodiments, at least 50% of the total number of hydrogen and fluorine atoms are fluorine atoms in the fluorinated monomer. In certain embodiments, at least 80% of the total number of hydrogen and fluorine atoms are fluorine atoms in the fluorinated monomer. In certain embodiments, at least 90% of the total number of hydrogen and fluorine atoms are fluorine atoms in the fluorinated monomer. In certain embodiments, the fluorinated monomer is perfluorinated (i.e., all hydrogen atoms, or at least all non-exchangeable hydrogen atoms, are replaced with fluorine atoms).

As described above, the hair and cosmetic treatments may include a polymerization initiator that initiates activation (i.e., polymerization) of the monomers and the short polymers in the hair or cosmetic treatment. A variety of activatable polymerization initiators may be used in the present invention, depending upon the polymerizable monomer and activation means for a selected treatment. In particular, appropriate initiators must be compatible with, i.e., capable of initiating polymerization of, at least one of the one or more polymerizable monomers used in the treatment. In addition, appropriate initiators must be capable of activation using the selected activation method. For example, the initiator is a photo-activated initiator if the selected activation means is light, or a heat-activated initiator if the selected activation means is heat. In certain embodiments, the activation means is visible light and the polymerization initiator is a titanocene.

The methods and compositions can utilize one or more initiators, for example, two or more, or three or more initiators. In one embodiment, more than one initiator is included, and in certain particular embodiments, each of the multiple initiators initiates the polymerization of a different monomer used in the treatment.

In one embodiment, at least one polymerization initiator in the composition is oxygen tolerant. In a preferred embodiment, at least one PI in the composition is oxygen tolerant and is selected from 4,4′-azobis(4-cyanovaleric acid); 1,1′-azobis(cyclohexanecarbonitrile); 2,2′-azobis(2-methylpropionitrile); benzoyl peroxide; 2,2-bis(tert-butylperoxy)butane; 2,5-bis(tert-butylperoxy)-2,5-dimethylhexane; bis[1-(tert-butylperoxy)-1-methylethyl]benzene; tert-butyl hydroperoxide; tert-butyl peracetate; tert-butyl peroxide; tert-butyl peroxybenzoate; cumene hydroperoxide; dicumyl peroxide; lauroyl peroxide; peracetic acid; potassium persulfate; 2-hydroxy-2-methyl-phenylpropanone; 2,4,6-trimethylbenzoyldiphenyl phosphine oxide; 2,4,6-trimethyl benzophenone; oligo(2-hydroxy-2-methyl-1-(4-(1-methylvinyl)phenyl)propanone and 4-methylbenzophenone.

In another embodiment, at least one polymerization initiator in the composition is a free radical initiator. In a preferred embodiment, the free radical initiator can include, but is not limited to, an initiator selected from the group consisting of acetophenone; diphenyl(2,4,6-trimethylbenzoyl)phosphine; 4,4′-dimethoxybenzoin; anthraquinone; anthraquinone-2-sulfonic acid sodium salt; benzene-chromium(0)tricarbonyl; 4-(boc-aminomethyl)phenyl isothiocyanate; benzil; benzoin; benzoin ethyl ether; benzoin isobutyl ether; benzoin methyl ether; benzophenone; benzoic acid; benzophenone/1-hydroxycyclohexyl phenyl ketone, 50/50 blend; benzophenone-3,3′,4,4′-tetracarboxylic dianhydride; 4-benzoylbiphenyl; 2-benzyl-2-(dimethylamino)-4′-morpholinobutyrophenone; 4,4′-bis(diethylamino)benzophenone; michler's ketone; (±)-camphorquinone; 2-chlorothioxanthen-9-one; 5-dibenzosuberenone; 2,2-diethoxyacetophenone; 4,4′-dihydroxybenzophenone; 2,2-dimethoxy-2-phenylacetophenone; 4-(dimethylamino)benzophenone; 4,4′-dimethylbenzil; 3,4-dimethylbenzophenone; diphenyl(2,4,6-trimethylbenzoyl)phosphine oxide; 2-hydroxy methylpropiophenone; 4′-ethoxyacetophenone; 2-ethylanthraquinone; ferrocene; 3′-hydroxyacetophenone; hydroxyacetophenone; 3-hydroxybenzophenone; 4-hydroxybenzophenone; 1-hydroxycyclohexyl phenyl ketone; 2-hydroxy-2-methylpropiophenone; 2-methylbenzophenone; 3-methylbenzophenone; methyl benzoylformate; 2-methyl-4′-(methylthio)-2-morpholinopropiophenone; 9,10-phenanthrenequinone; 4′-phenoxyacetophenone; thioxanthen-9-one; triarylsulfonium hexafluorophosphate salts, mixed 50% in propylene carbonate; 3-mercapto-1-propanol; 11-mercapto-1-undecanol; 1-mercapto-2-propanol and 3-mercapto-2-butanol. In a more preferred embodiment, the free radical initiator is selected from the group consisting of benzophenone, benzyl dimethyl ketal, 2-hydroxy-2-methyl-phenylpropanone; 2,4,6-trimethylbenzoyldiphenyl phosphine oxide; 2,4,6-trimethyl benzophenone; oligo(2-hydroxy-2-methyl-1-(4-(1-methylvinyl)phenyl)propanone and 4-methylbenzophenone.

In another embodiment, at least one polymerization initiator in the composition is a cationic radical initiator. In a preferred embodiment, the cationic radical initiator is selected from the group consisting of diaryliodonium and triarylsulfonium salts.

In another embodiment, at least one polymerization initiator in the composition is a thermal initiator. In a preferred embodiment, the thermal initiator can include, but is not limited to, an initiator selected from the group consisting of 4,4′-azobis(4-cyanovaleric acid); 1,1′-azobis(cyclohexanecarbonitrile); 2,2′-azobis(2-methylpropionitrile); benzoyl peroxide; 2,2-bis(tert-butylperoxy)butane; 2,5-bis(tert-butylperoxy)-2,5-dimethylhexane; bis[1-(tert-butylperoxy)-1-methylethyl]benzene; tert-butyl hydroperoxide; tert-butyl peracetate; tert-butyl peroxide; tert-butyl peroxybenzoate; cumene hydroperoxide; dicumyl peroxide; lauroyl peroxide; peracetic acid; and potassium persulfate. In a more preferred embodiment, the thermal initiator is selected from the group consisting of benzoyl peroxide and 2,2′-azo-bis-isobutyrylnitrile.

As described above, the monomers, oligomers, or polymers, and the polymerization initiators are mixed with an acceptable solvent to facilitate mixture of the monomers, oligomer, or polymers, and polymerization initiator and/or to facilitate application to the hair or skin. In preferred embodiments, the solvent is a pharmaceutically or physiologically acceptable solvent, and particularly is cosmetically acceptable. The solvent can comprise a single component as described further below or known in the art or can comprise a mixture of multiple solvents in appropriate proportions as known to the skilled artisan. Selection of an acceptable solvent will depend on the monomer and polymerization initiator to be used as well as the method of application of the hair or cosmetic treatment composition. An acceptable solvent should not adversely impact the in situ polymerization process.

In certain embodiments, the monomers and/or polymerization initiator is soluble in water or an aqueous solution. An aqueous solution may be acid or basic. In certain embodiments, the monomer and/or polymerization initiator is soluble in an alcohol (e.g., methanol, ethanol, denatured ethanol, isopropanol, butanol). Examples of solvents for use in delivering the monomers and/or polymerization initiators include, but are not limited to, solvents selected from the group consisting of acetic acid, acetone, alcohol, alcohol (denatured), benzophenone, butoxydlglycol, butyl acetate, n-butyl acetate, n-butyl alcohol, butylene glycol, butyl myristate, butyloctyl benzoate, butyloctyl salicylate, butyl stearate, C12-15 alkyl benzoate, capric acid, caprylic alcohol, cetearyl octanoate, cetyl stearyl octanoate, chlorobutanol, C9-11 isoparaffin, C10-11 isoparaffin, C10-13 isoparaffin, decyl alcohol, diethylene glycol, diethylene glycol dibenzoate, diethylhexyl maleate, diethylhexyl 2,6-naphthalate, diethyl sebacate, diisocetyl adipate, diisopopyl adipate, diifiopropyl sebacate, dimethylphthalate, dioctyl adipate, dioctyl succinate, dipropylene glycol, dipropylene glycol dibenzoate, ethoxydiglycol, ethyl acetate, ethyl lactate, ethyl macadamiate, ethyl myristate, ethyl oleate, glycereth-7 benzoate, glycereth-7-diisononanoate, glycereth-4,5-lactate, glycereth-7 triacetate, glycerin, glycine soja (soybean) oil, glycofurol, heptane, hexyl alcohol, hexyldecyl benzoate, hexylene glycol, isobutyl stearate, isocetyl salicylate, isodecyl benzoate, isodecyl isononanoate, isodecyl octanoate, isodecyl oleate, isododecane, isoeicosane, isohexadecane, isononyl isononanoate, isooctane, isopropyl alcohol, isopropyl laurate, isopropyl myristate, isopropyl palmitate, isostearyl stearoyl stearate, laneth-5, lanolin oil, laureth-2 acetate, MEK, methoxydiglycol, methyl acetate, methyl alcohol, methylene chloride, methylpropanediol, methylsoyate, MIBK, morpholine, neopentyl glyol, neopentyl glyol dioctanoate, nonocynol-9, octyl benzoate, octyldodecyl lactate, octyldodecyl octyldodecanoate, octyl isononanoate, octyl isostearate, octyl laurate, octyl palmitate, octyl stearate, oleyl alcohol, olive oil PEG-6 esters, peanut pil PEG-6 esters, PEG-12, PBG-33 castor oil, PEG-50 glyceryl cocoate, PEG-20 hydrogenated castor oil, PEG-6 methyl ether, penetaerythrity tetracaprylate/tetracaprate, pentane, petroleum distillates, polyglyceryl-3 diisostearate, polyglyceryl-2 dioleate, polyoxyethylene glycol dibenzoate, PPG-3, PPG-20 lanolin alcohol ether, PPG-2 myristyl ether propionate, propyl alcohol, propylene carbonate, propylene glycol, propylene glycol caprylate, propylene glycol dibenzoate, propylene glycol methyl ether, propylene glycol myristate, pyridine, ricinus communis (castor) seed oil, sesamum indicum (sesame) oil, sorbitan trioleate, stearyl heptaroate, toluene, 2,2,4-timethylpentane, xylene. In a preferred embodiment, the solvent is selected from the group consisting of propylene glycol, ethanol, isopropanol, n-butanol, water and mixtures thereof. In certain embodiments, the solvent comprises denatured ethanol. In a certain particular embodiment, the solvent comprises a mixture of propylene glycol and denatured ethanol.

The monomer and polymerization initiator can be added concurrently or sequentially to a solvent, or each of the monomer and the polymerization initiator can be added to a separate solvent, and subsequently the separate solvents can be combined. In this embodiment, the separate solvent compositions can be the same or different. Preferably, the solvent systems are miscible, and mixing the two solutions does not cause either the monomer or polymerization initiator to precipitate out of solution.

Alternatively, each of the monomer and the polymerization can be separately applied to the hair or skin. For example, the monomer can be mixed with a solvent and the polymerization initiator can be mixed with a separate solvent; the solvents can be the same or different. The monomer/solvent mixture and the polymerization initiator/solvent mixture can then be sequentially applied to the hair or skin in any order. In all embodiments, the solvent is optional and need not be present along with either the monomer or the polymerization initiator, depending on the properties of either.

### Other Embodiments

The foregoing has been a description of certain non-limiting embodiments of the invention. Those of ordinary skill in the art will appreciate that various changes and modifications to this description may be made without departing from the spirit or scope of the present invention, as defined in the following claims.

1. A method for treating hair or skin, comprising:
providing a solution that includes at least one polymerizable monomer and optionally a polymerization initiator,
applying the solution to the hair or skin, and
polymerizing by an external activator the monomers of the solution.

2. The method of claim 1, wherein the step of polymerizing by an external activator comprises polymerizing the polymerizable monomers using a light source.

3. The method of claim 1, wherein the step of polymerizing by a light source comprises polymerizing the polymerizable monomers using an ultraviolet light source.

4. The method of claim 1, wherein the step of polymerizing by a light source comprises polymerizing the polymerizable monomers using a visible light source.

5. The method of claim 1, wherein the step of polymerizing by a light source comprises polymerizing the polymerizable monomers using an infrared light source.

6. The method of claim 1, wherein the step of polymerizing by an external activator comprises polymerizing the polymerizable monomers using a light source that produces light of wavelengths between about 200 mm and about 600 nm n.

7. The method of claim 1, wherein the step of polymerizing by an external activator comprises polymerizing the polymerizable monomers using a light source that produces light of wavelengths between about 200 nm and about 400 nm.

8. The method of claim 2, wherein the light source is selected from the group consisting of mercury-based sources, xenon-based sources, deuterium-based sources, metal-halide-based sources, phosphorescent-based sources, incandescent-based sources, and LED-based sources.

9. The method of claim 1, wherein the step of polymerizing by an external activator comprises polymerizing the polymerizable monomers using a heat source.

10. The method of claim 9, wherein the step of polymerizing the polymerizable monomers using a heat source comprises polymerizing the polymerizable monomers using a heat source capable of delivering heat by convection.

11. The method of claim 9, wherein the step of polymerizing the polymerizable monomers using a heat source comprises polymerizing the polymerizable monomers using a heat source capable of delivering heat by radiation.

12. The method of claim 1, wherein the step of polymerizing by an external activator comprises:
wrapping the hair around an elongated tubular body,
clasping the hair about the elongate tubular body by a clasp,
optionally styling the hair, and
emitting using the elongated tubular body or the clasp light or heat sufficient to effect polymerization.

13. The method of claim 1, wherein the step of polymerizing by an external activator comprises:
disposing the hair on a planar surface of a body of a device,
clasping using a clasp the hair to the planar surface of the body using a planar surface of the clasp,
optionally styling the hair, and
emitting using the body and/or the clasp light and/or heat sufficient to effect polymerization.

14. The method of claim 1, wherein the step of polymerizing by an external activator comprises:
wrapping at least one tress of hair around an elongated cylindrical curler,
setting the curlers, and
emitting using the curler light and/or heat sufficient to effect polymerization.

15. The method of claim 1, wherein the step of polymerizing by an external activator comprises:
providing a device having an elongated body and a handle oriented at an angle to the elongate body,
pointing the elongate body towards a target region of the hair, and
emitting by the device at least one of light and/or heat sufficient to effect polymerization.

16. The method of claim 1, wherein the step of polymerizing by an external activator comprises:
providing a device having a handle, a body affixed to the handle, and a plurality of bristles emanating from a surface of the body,
running the bristles through the hair, and
emitting by the bristles or the surface the bristles are attached to light and/or heat sufficient to effect polymerization.

17. The method of claim 1, wherein the step of polymerizing by an external activator comprises:
placing finger covers about respective fingers on a hand of the user,
contacting the finger covers to the hair or skin, and
emitting by the finger covers light and/or heat sufficient to effect polymerization.

18. The method of claim 1, wherein the step of polymerizing by an external activator comprises:
disposing a head covering around the head of hair, and
emitting by the head covering light and/or heat sufficient to effect polymerization.

19. The method of claim 18, wherein disposing a head covering around the head comprises disposing a dome around at least an upper portion of the head.

20. The method of claim 1, wherein:
the step of applying the solution of monomers to the hair or skin comprises dispensing the solution from a dispenser, and
the step of polymerizing the monomers of the solution comprises emitting light and/or heat from the dispenser.

21. The method of claim 1, wherein the step of polymerizing by an external activator comprises:
isolating a tress of the hair,
encapsulating at least a portion of the tress of the hair by foil, and
emitting by the foil at least one of light and heat.

22. The method of claim 1, wherein the step of applying the solution to the hair or skin comprises at least one of pump spraying and aerosol spraying the solution onto the hair or skin.

23. The method of claim 1, wherein the step of applying the solution to the hair or skin comprises applying a gel with polymerizable monomers to the hair or skin.

24. The method of claim 1, wherein the step of applying the solution to the hair or skin comprises applying a foam with polymerizable monomers to the hair or skin.

25. The method of claim 1, wherein the step of providing a solution that includes polymerizable monomers comprises providing a solution that includes two or more polymerizable monomers.

26. The method of claim 1, wherein the step of applying the solution to the hair comprises applying the solution to eyelashes.

27. The method of claim 26, wherein:
the step of applying the solution to the eyelashes comprises applying the solution with an elongate wand with bristles emanating therefrom, and
the step of polymerizing by an external activator comprises emitting, by the bristles or the surface the bristles are attached to, at least one of light and heat sufficient to effect polymerization.

28. The method of claim 26, wherein the step of polymerizing by an external activator comprises:
interdisposing the eyelashes between an inner eyelash-contacting surface and an outer eyelash-contacting surface,
interfitting the outer eyelash-contacting surface against the inner eyelash-contacting surface to curve the eyelashes, and
emitting, by at least one of the inner eyelash-contacting surface and the outer eyelash-contacting surface, at least one of light and heat sufficient to effect polymerization.

29. The method of claim 1, further comprising:
emitting visible light, by a light source, to indicate regions of the hair or skin on which polymerization is occurring.

30.-125. (canceled)
